(19) United States
(12) Patent Application Publication
(10) Pub. No.: US 2026/0159683 A1
(43) Pub. Date: Jun. 11, 2026

(54) DISSOLVABLE SOLID STRUCTURE HAVING FIRST AND SECOND LAYERS

(71) Applicant: THE PROCTER & GAMBLE COMPANY, Cincinnati, OH (US)

(72) Inventors: Mark William HAMERSKY, Hamilton, OH (US); Jennifer Elaine HILVERT, Cincinnati, OH (US); Min MAO, Deerfield, MI (US); Dinah Achola NYANGIRO, Cincinnati, OH (US)

(21) Appl. No.: 19/428,760

(22) Filed: Dec. 22, 2025

Related U.S. Application Data
(63) Continuation of application No. 18/181,675, filed on Mar. 10, 2023, now Pat. No. 12,540,238.
(60) Provisional application No. 63/318,395, filed on Mar. 10, 2022.

(51) Int. Cl. C08L 29/04; C08K 5/1545; C08K 5/16

(52) U.S. Cl. CPC C08L 29/04; C08K 5/1545; C08K 5/16; C08K 2201/014

(57) ABSTRACT

A dissolvable solid structure with a first layer and a second layer. The first layer includes from about 20% to about 100%, by weight of the first layer, of a first water-soluble polymer. The second layer includes from about 1% to about 50%, by weight of the second layer, of a second water-soluble polymer, a high melting point fatty material having an average carbon chain length C12-C22 and a melting point above 25° C., and a cationic surfactant. The dissolvable solid structure can provide improved flexibility and solubility in a composition, such as water, while having a sufficient structural integrity during manufacturing.

### FIELD OF THE INVENTION

The present invention relates to A dissolvable solid structure comprising: (a) a first layer comprising from about 20% to about 100%, by weight of the first layer by weight of the first layer of a first water-soluble polymer; and (b) b second layer comprising: from about 1% to about 50%, by weight of the second layer, of a second water-soluble polymer; a high melting point fatty material having a carbon chain length C12-C22 or mixtures thereof, wherein the melting point is above 25° C.; and a cationic surfactant. The present invention provides for example, improved flexibility in a composition, while keeping a certain structural strength during manufacturing.

### BACKGROUND OF THE INVENTION

Many personal care and other consumer products in the market today are sold in liquid form. While widely used, liquid products often have tradeoffs in terms of packaging, storage, transportation, and convenience of use. Liquid consumer products typically are sold in bottles which add cost as well as packaging waste, much of which ends up in land-fills.

Hair Care products in the form of a dissolvable solid structures present an attractive form to consumers. Market executions of dissolvable solid structures may include, dissolvable films, compressed powders in a solid, fibrous structures, porous foams, soluble deformable solids, powders, etc.

However, a need still exists for such dissolvable solid structures which deliver at least one of the followings:
Improved flexibility in a composition, especially allowing its lower contents of polymers at least in one of the layers, while keeping a certain structural strength during manufacturing and easiness to cut during and after manufacturing
Allowing incorporation of ingredients in one layer, which are incompatible to at least one of ingredients contained in another layer,
Allowing improved corporation of particulates in the article
Improved aesthetic feature selected from the group consisting of printing, embossing, texture, colored and mixtures thereof.

### SUMMARY OF THE INVENTION

The present invention is directed to a dissolvable solid structure comprising:
a. A first layer comprising from about 20% to about 100%, by weight of the first layer by weight of the first layer of a first water-soluble polymer;
b. A second layer comprising: from about 1% to about 50%, by weight of the second layer, of a second water-soluble polymer; a high melting point fatty material having a carbon chain length C12-C22 or mixtures thereof, wherein the melting point is above 25° C.; and a cationic surfactant.

### BRIEF DESCRIPTION OF THE DRAWINGS

FIG. 1 is a schematic representation of an example of a process for making fibrous elements of the present invention;

FIG. 2 is a schematic representation of an example of a die with a magnified view used in the process of FIG. 1;

FIG. 3 is a schematic representation of an example of a process for making an example of a fibrous structure according to the present disclosure;

FIG. 4 is a schematic representation of an example of a die with a magnified view used in the process of FIG. 3; and

FIG. 5 is a schematic representation of a cross-sectional view of another example of a fibrous structure according to the present disclosure.

### DETAILED DESCRIPTION OF THE INVENTION

### Definitions

As used herein, The Dissolvable Solid Structure may be referred to herein as “the Dissolvable Solid Structure”, “the Structure”, or “the Dissolvable Structure”.

As used herein, “dissolvable” means that the Dissolvable Solid Structure is completely soluble in water or it provides a uniform dispersion upon mixing in water according to the hand dissolution test. The Dissolvable Solid Structure has a hand dissolution value of from about 1 to about 30 strokes, alternatively from about 2 to about 25 strokes, alternatively from about 3 to about 20 strokes, and alternatively from about 4 to about 15 strokes, as measured by the Hand Dissolution Method.

As used herein, “flexible” means a Dissolvable Solid Structure meets the distance to maximum force values discussed herein.

“Fibrous structure” as used herein means a structure that comprises one or more fibrous elements and optionally, one or more particles. The fibrous structure as described herein can mean an association of fibrous elements and optionally, particles that together form a structure, such as a unitary structure, capable of performing a function.

The fibrous structure may have a multi-ply fibrous structure which comprises two or more different fibrous structure plies. Each ply may be the same as or different from other ply.

A layer comprising fibrous elements may sometimes be referred to as a ply. A ply may be a fibrous structure which may be homogeneous or layered as described herein.

The single-ply fibrous structure or a multi-ply fibrous structure comprising one or more fibrous structure plies as described herein may exhibit a basis weight of less than 5000 g/m² as measured according to the Basis Weight Test Method described herein. For example, the single- or multi-ply fibrous structure according to the present invention may exhibit a basis weight of greater than 10 g/m² to about 5000 g/m² and/or greater than 10 g/m² to about 3000 g/m² and/or greater than 10 g/m² to about 2000 g/m² and/or greater than 10 g/m² to about 1000 g/m² and/or greater than 20 g/m² to about 800 g/m² and/or greater than 30 g/m² to about 600 g/m² and/or greater than 50 g/m² to about 500 g/m² and/or greater than 300 g/m² to about 3000 g/m² and/or greater than 500 g/m² to about 2000 g/m² as measured according to the Basis Weight Test Method.

In one example, the fibrous structure is a “unitary fibrous structure.”

“Unitary fibrous structure” as used herein is an arrangement comprising a plurality of two or more and/or three or more fibrous elements that are inter-entangled or otherwise associated with one another to form a fibrous structure and/or fibrous structure plies. A unitary fibrous structure may be one or more plies within a multi-ply fibrous structure. In one example, a unitary fibrous structure may comprise three or more different fibrous elements. In another example, a unitary fibrous structure may comprise two or more different fibrous elements.

“Article” as used herein refers to a consumer use unit, a consumer unit dose unit, a consumer use saleable unit, a single dose unit, or other use form comprising a unitary fibrous dissolvable solid structure and/or comprising one or more fibrous structures.

“Fibrous element” as used herein means an elongated particulate having a length greatly exceeding its average diameter, i.e., a length to average diameter ratio of at least about 10. A fibrous element may be a filament or a fiber. In one example, the fibrous element is a single fibrous element rather than a yarn comprising a plurality of fibrous elements.

The fibrous elements may be spun from a filament-forming compositions also referred to as fibrous element-forming compositions via suitable spinning process operations, such as meltblowing, spunbonding, electro-spinning, and/or rotary spinning.

The fibrous elements may be monocomponent (single, unitary solid piece rather than two different parts, like a core/sheath bicomponent) and/or multicomponent. For example, the fibrous elements may comprise bicomponent fibers and/or filaments. The bicomponent fibers and/or filaments may be in any form, such as side-by-side, core and sheath, islands-in-the-sea and the like.

“Filament” as used herein means an elongated particulate as described above that exhibits a length of greater than or equal to 5.08 cm (2 in.) and/or greater than or equal to 7.62 cm (3 in.) and/or greater than or equal to 10.16 cm (4 in.) and/or greater than or equal to 15.24 cm (6 in.). Filaments are typically considered continuous or substantially continuous in nature. Filaments are relatively longer than fibers. Non-limiting examples of filaments include meltblown and/or spunbond filaments.

“Fiber” as used herein means an elongated particulate as described above that exhibits a length of less than 5.08 cm (2 in.) and/or less than 3.81 cm (1.5 in.) and/or less than 2.54 cm (1 in.). Fibers are typically considered discontinuous in nature. Non-limiting examples of fibers include staple fibers produced by spinning a filament or filament tow and then cutting the filament or filament tow into segments of less than 5.08 cm (2 in.) thus producing fibers. Therefore, references to filaments herein also include fibers made from such filaments unless otherwise noted. Fibers are typically considered discontinuous in nature relative to filaments, which are considered continuous in nature.

“Filament-forming composition” and/or “fibrous element-forming composition” as used herein means a composition that is suitable for making a fibrous element such as by meltblowing and/or spunbonding. The filament-forming composition comprises one or more filament-forming materials that exhibit properties that make them suitable for spinning into a fibrous element. In one example, the filament-forming material comprises a polymer. In addition to one or more filament-forming materials, the filament-forming composition may comprise one or more additives, for example one or more active agents. In addition, the filament-forming composition may comprise one or more polar solvents, such as water, into which one or more, for example all, of the filament-forming materials and/or one or more, for example all, of the active agents are dissolved and/or dispersed prior to spinning a fibrous element, such as a filament from the filament-forming composition.

As used herein, “porous” means that the Dissolvable Solid Structure has spaces, voids or interstices, (generally referred to herein as “pores”) provided by the microscopic complex three-dimensional configuration, that provide channels, paths or passages through which a liquid can flow.

As used herein, “porosity” and “percent porosity” are used interchangeably and each refers to a measure of void volume of the Dissolvable Solid Structure and is calculated as

[1−([basis weight of Dissolvable Solid Structure]/[thickness of Dissolvable Solid Structure×density of the bulk, dried material])]×100%

with the units adjusted so they cancel and multiplied by 100% to provide percent porosity.

The Dissolvable Solid Structure may be referred to herein as “the Dissolvable Solid Structure” or “the Dissolvable Structure”.

The term “molecular weight” or “Molecular weight” refers to the weight average molecular weight unless otherwise stated. Molecular weight is measured using industry standard method, gel permeation chromatography (“GPC”).

As used herein, the articles including “a” and “an” when used in a claim, are understood to mean one or more of what is claimed or described.

As used herein, the terms “include,” “includes,” and “including,” are meant to be non-limiting.

The methods disclosed in the Test Methods Section of the present application should be used to determine the respective values of the parameters of Applicants' inventions, including those discussed in the Dissolvable Structures-Physical Characteristics section below.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Dissolvable Solid Structure (Dissolvable Solid Article)

The dissolvable solid structure can comprise: a first layer comprising a first water-soluble polymer; and a second layer comprising a second water-soluble polymer. Details of each layer and polymer are described later.

In one embodiment of the above dissolvable solid structure, the first layer may comprise: the first water-soluble polymer; and an ingredient incompatible to at least one of the above ingredients in the second layer, and wherein the second layer may comprise: the second water-soluble polymer; a high melting point fatty material having a carbon chain length C12-C22 or mixtures thereof, wherein the melting point is above 25° C.; and a cationic surfactant.

In one embodiment, in the dissolvable solid structure of any of the above features, at least one of the first and second layers may further comprise a particulate, alternatively the second layer may further comprise a particulate.

In one embodiment, in the dissolvable solid structure of any of the above features, the first layer may have an aesthetic feature selected from the group consisting of printing, embossing, texture, colored and mixtures thereof.

The dissolvable solid structure can be a personal care product, such as a hair care product, which can be a rinse-off hair care product that can include a non-sulfate surfactant.

### First Layer and First Water Soluble Polymer

The first layer comprises from about 20% to about 100%, alternatively from about 50% to about 100%, alternatively from about 90% to about 100%, alternatively from about 95% to about 100%, alternatively from about 99% to about 100%, by weight of the first layer, of a first water-soluble polymer.

The first water-soluble polymer can be natural or synthetic polymers, provided the first layer has a Scaled Fail Tensile Energy Absorption (Scaled Fail TEA) value described below. In other words, the first water-soluble polymer can be any polymer, as long as such polymers can be converted into a fibrous layer; and the layer can have a sufficient strength to permit processing latter stage of manufacturing process, i.e., the layer exhibits adequate strength to support the second layer during manufacturing, while allowing cut of such layer or article

Alternatively, the first water-soluble polymer is selected from the group consisting of:
(i) a polyvinyl alcohol having a molecular weight of from about 23,000 g/mol to about 45,000 g/mol, alternatively from about 25,000 g/mol to about 40,000 g/mol, alternatively from about 26,000 g/mol to about 35,000 g/mol;
(ii) a mixture of polyvinyl alcohols, wherein the mixture has an average molecular weight as a mixture of from about 23,000 g/mol to about 45,000 g/mol, alternatively from about 25,000 g/mol to about 40,000 g/mol, alternatively from about 26,000 g/mol to about 35,000 g/mol;
(iii) A thermal bonded starch with or without a plasticizer, wherein molecular weight of from about 1,000,000 g/mol to about 50,000,000 g/mol, alternatively from about 2,000,0000 g/mol to about 40,000,000 g/mol;
(iv) Polyvinylpyrrolidone, its copolymer, or mixtures thereof (for example, that having a tradename of PVP K60 from Ashland, Inc., which have a MW of around 400,000 g/mol);
(v) Poly vinyl oxazoline, its copolymer, or mixtures thereof;
(vi) Poly 2-ethyl-2oxazoline, its copolymer, or mixtures thereof; and
(vii) Polyvinylcaprolactam, its copolymer, or mixtures thereof.

Among them, more preferred first water-soluble polymer is selected from (ii) the mixture of polyvinyl alcohols.

The first water soluble polymer in the first layer can be same or different from the second polymer in the second layer.

As used in the present invention, the term “water-soluble polymer” is broad enough to include both water-soluble and water-dispersible polymers, and is defined as a polymer with a solubility in water, measured at 25° C., of at least about 0.1 gram/liter (g/L). In some embodiments, the polymers have a solubility in water, measured at 25° C., of from about 0.1 gram/liter (g/L). to about 500 grams/liter (g/L). (This indicates production of a macroscopically isotropic or transparent, colored or colorless solution). The polymers for making these solids may be of synthetic or natural origin and may be modified by means of chemical reactions. They may or may not be film-forming. These polymers should be physiologically acceptable, i.e., they should be compatible with the skin, mucous membranes, the hair and the scalp.

The terms “water-soluble polymer” and “polymer structurant” are used interchangeably herein. Furthermore, whenever the singular term “polymer” is stated, it should be understood that the term is broad enough to include one polymer or a mixture of more than one polymer. For instance, if a mixture of polymers is used, the polymer solubility as referred to herein would refer to the solubility of the mixture of polymers, rather than to the solubility of each polymer individually.

Alternatively, the first layer is substantially free of a surfactant, especially when the first layer has an aesthetic feature selected from the group consisting of printing, embossing, texture, colored and mixtures thereof. In the present invention, “the layer being substantially free of surfactants” means that: the composition is free of surfactants; or, if the composition contains surfactants, the level of such surfactants is very low. In the present invention, a total level of such surfactants, if included, alternatively 1% or less, alternatively 0.5% or less, alternatively 0.1% or less by weight of the composition. Most alternatively, the total level of such surfactants is 0% by weight of the composition. Surfactants herein means any type of ionic surfactants such as anionic, cationic, amphoteric and zwitterionic surfactants. Surfactants herein also include nonionic surfactants having HLB of above 9 (excluding 9).

The first layer may further comprise oily components having HLB of 9 or less, alternatively 3 or less, such as oleic acid. The oily components can be included in the first layer at a level by weight of the first layer, from about 0.01% to about 2%, alternatively from about 0.1% to about 1%.

### Scaled Fail TEA

It may be preferred that the first layer has a Scaled Fail Tensile Energy Absorption (Scaled Fail TEA) of from about 10 (g/cm) to about 185 (g/cm), alternatively form about 20 (g/cm) to about 180 (g/cm), alternatively from about 25 (g/cm) to about 175 (g/cm).

Scaled Fail TEA is a Fail TEA which has been scaled to a common basis weight, i.e., 50 gsm herein. This allows comparisons of the strengths of different basis weight materials, since the Fail TEA is dependent on the amount of materials present either in thickness or cross sectional area. The method for measuring Fail TEA is shown below.

### The Method for Measuring Fail TEA

Sample prep: A steel rule die on a 12″×12″ plywood base stamps out the strips from the material Equilibration: 73 F & 50% RH for at least 15 minutes.
Crosshead speed: 2 in/min
Sample width: 1 in
Gauge length: 1 in
Data analysis: Fail TEA (area under-the-curve at the fail point, as outlined in fail stretch).

### Second Layer and Second Water Soluble Polymer

The second layer comprises from about 1% to about 50%, alternatively from about 1% to about 20%, alternatively from about 1% to about 10%, alternatively from about 2% to about 6%, alternatively from about 3% to about 5%, by weight of the second layer, of a second water-soluble polymer.

The second-soluble polymer can be any polymer, and alternatively different from the first polymer in the first layer. The structurant has a weight average molecular weight of from about 10,000 to about 6,000,000 g/mol. The structurant having a weight average molecular weight of from about 3,000,000 g/mol to about 5,000,000 g/mol in included at a level of from about 3 wt % to about 6 wt %. Alternatively, a structurant having a weight average molecular weight of from about 50,000 g/mol to about 100,000 g/mol can be included at a level of from about 30 wt % to about 50 wt %.

Alternatively, the second water-soluble polymer is selected from the group consisting of: polyvinylpyrrolidone, alternatively a polyvinylpyrrolidone copolymer; polydimethylacrylamide, alternatively a polydimethylacrylamide copolymer; a poly(vinyl oxazoline), alternatively poly 2-ethyl-2-oxazoline; a polyvinylcaprolactam copolymer, preferable polyvinyl caprolactam and combinations thereof

As used herein, “vinyl pyrrolidone copolymer” (and “copolymer” when used in reference thereto) refers to a polymer of the following structure (I):

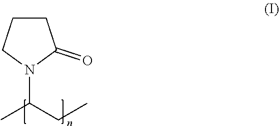

In structure (I), n is an integer such that the polymeric structurant has the degree of polymerization such that it possesses characteristics described herein. For purposes of clarity, the use of the term “copolymer” is intended to convey that the vinyl pyrrolidone monomer can be copolymerized with other non-limiting monomers such as vinyl acetate, alkylated vinyl pyrrolidone, vinyl oxazoline, vinyl caprolactam, vinyl valerolactam, vinyl imidazole, acrylic acid, methacrylate, acrylamide, methacrylamide, dimethacrylamide, alkylaminomethacrylate, and alkylaminomethacrylamide monomers.

For example, suitable polymers for use are PVP K120 from Ashland Inc., having a weight average molecular weight of about 3,500,000 g/mol is soluble in the oil and water and enables fibers to be formed and collected onto a belt. Additional suitable polymers include copolymers of polyvinylpyrrolidone, such as Ganex® or PVP/VA (weight average molecular weight of about 50,000 g/mol) copolymers from Ashland Inc., also performed as suitable structurants but a higher level was utilized to be effective due to their lower weight average molecular weight. Additional polymers that are suitable include poly(2-ethyl-2-oxazoline), polyvinyl caprolactam, and polydimethylacrylamide and their copolymers.

### Dispersing Agents

The second layer may further comprise a dispersing agent for increasing the wetting, hydration, and/or dispersion of the conditioner materials. The dispersing agent can be included at a level of from about 1 wt % to about 30 wt %, alternatively from about 5 wt % to about 15 wt %, and alternatively from about 5 wt % to about 10 wt % by weight of the second layer. A surfactant from the nonionic class of alkyl glucamides can improve the wetting and hydration when added to the solid conditioner formula. The alkyl glucamide surfactant contains a hydrophobic tail of about 8-18 carbons and a nonionic head group of glucamide. For glucamide, the presence of the amide and hydroxyl groups may provide sufficient polarity that balances the hydrophobic carbon tail in such a way to permit the surfactant's solubility in the conditioner oils and also imparts a rapid dispersion of the conditioner ingredients upon exposure to water. Other similar dispersing agents include, but are not limited to, reverse alkyl glucamides, gluconamides, cocoamiodpropyl betaines, alkyl glucoside, triethanol amine, cocamide MEAs and mixtures thereof.

The alkyl glucamide surfactants, especially when used together with polyvinylpyrrolidone, may provide styling benefit. Such benefit of alkyl glucamide surfactants might be seen regardless of the product forms, i.e., not only from a solid form such as dissolvable solid structures, but also from a liquid form.

### Cationic Surfactant

The second layer comprise a cationic surfactant which can be included at a level of from about 1 wt % to about 60 wt %, alternatively from about 10 wt % to about 50 wt %, alternatively from about 20 wt % to about 40 wt % by weight of the second layer.

Cationic surfactant useful herein can be one cationic surfactant or a mixture of two or more cationic surfactants. The cationic surfactant can be selected from the group consisting of, but not limited to: a mono-long alkyl quaternized ammonium salt; a combination of a mono-long alkyl quaternized ammonium salt and a di-long alkyl quaternized ammonium salt; a mono-long alkyl amine; a combination of a mono-long alkyl amine and a di-long alkyl quaternized ammonium salt; and a combination of a mono-long alkyl amine and a mono-long alkyl quaternized ammonium salt, a tertiary amine and combinations thereof.

### Mono-Long Alkyl Amine

Mono-long alkyl amine useful herein are those having one long alkyl chain of from 12 to 30 carbon atoms, alternatively from 16 to 24 carbon atoms, alternatively from 18 to 22 alkyl group. Mono-long alkyl amines useful herein also include mono-long alkyl amidoamines. Primary, secondary, and tertiary fatty amines are useful.

Suitable for use in the dissolvable solid structure are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al.

These amines can be used in combination with acids such as 1-glutamic acid, lactic acid, hydrochloric acid, malic acid, succinic acid, acetic acid, fumaric acid, tartaric acid, citric acid, 1-glutamic hydrochloride, maleic acid, and mixtures thereof; alternatively 1-glutamic acid, lactic acid, citric acid, at a molar ratio of the amine to the acid of from about 1:0.3 to about 1:2, alternatively from about 1:0.4 to about 1:1.

### Mono-Long Alkyl Quaternized Ammonium Salt

The mono-long alkyl quaternized ammonium salts useful herein are those having one long alkyl chain which has from 12 to 30 carbon atoms, alternatively from 16 to 24 carbon atoms, alternatively a C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms. Mono-long alkyl quaternized ammonium salts useful herein are those having the formula (I):

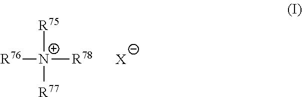

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, alternatively from 16 to 24 carbon atoms, alternatively from 18 to 22 carbon atoms, alternatively 22 carbon atoms; the remainder of R⁷⁵, R⁷⁶, R⁷⁷ and R⁷⁸ can be independently selected from CH₃, C₂H₅, C₂H₄OH, and mixtures thereof, and X can be selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Di-Long Alkyl Quaternized Ammonium Salts

When used, di-long alkyl quaternized ammonium salts can be combined with a mono-long alkyl quaternized ammonium salt and/or mono-long alkyl amine salt, at the weight ratio of from 1:1 to 1:5, alternatively from 1:1.2 to 1:5, alternatively from 1:1.5 to 1:4, in view of stability in rheology and conditioning benefits.

Di-long alkyl quaternized ammonium salts useful herein are those having two long alkyl chains of from 12 to 30 carbon atoms, alternatively from 16 to 24 carbon atoms, alternatively from 18 to 22 carbon atoms. Such di-long alkyl quaternized ammonium salts useful herein are those having the formula (I):

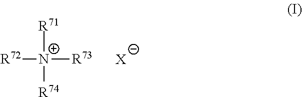

wherein two of R⁷¹, R⁷², R⁷³ and R⁷⁴ are selected from an aliphatic group of from 12 to 30 carbon atoms, alternatively from 16 to 24 carbon atoms, alternatively from 18 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, alternatively from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Two of R⁷¹, R⁷², R⁷³ and R⁷⁴ can be selected from an alkyl group of from 12 to 30 carbon atoms, alternatively from 16 to 24 carbon atoms, alternatively from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Suitable di-long alkyl cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### High Melting Point Fatty Compound

The second layer comprises a high melting point fatty compound. The high melting point fatty compound can be included at a level of from about 10 wt % to about 85 wt %, alternatively from 20 wt % to 70 wt %, alternatively from about 50 wt % to about 70 wt %, alternatively from about 10 wt % to about 20 wt % by weight of the second layer. The fatty compound can be selected from the group consisting of, but not limited to, fatty amphiphiles, fatty alcohol, fatty acid, fatty amide, fatty ester and combinations thereof.

The high melting point fatty compound useful herein have a melting point of 25° C. or higher, alternatively 40° C. or higher, alternatively 45° C. or higher, alternatively 50° C. or higher, in view of stability of the emulsion especially the gel matrix. Such melting point is up to about 90° C., alternatively up to about 80° C., alternatively up to about 70° C., alternatively up to about 65° C., in view of easier manufacturing and easier emulsification. The high melting point fatty compound can be used as a single compound or as a blend or mixture of at least two high melting point fatty compounds. When used as such blend or mixture, the above melting point means the melting point of the blend or mixture.

The high melting point fatty compound useful herein is selected from the group consisting of fatty alcohols, fatty acids, fatty alcohol derivatives, fatty acid derivatives, fatty amides, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than the above. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols can be used in the composition described herein. The fatty alcohols useful herein are those having from about 14 to about 30 carbon atoms, alternatively from about 16 to about 22 carbon atoms. These fatty alcohols are saturated and can be straight or branched chain alcohols.

Suitable fatty alcohols include, but are not limited to, cetyl alcohol (having a melting point of about 56° C.), stearyl alcohol (having a melting point of about 58-59° C.), behenyl alcohol (having a melting point of about 71° C.), and mixtures thereof. These compounds are known to have the above melting point. However, they often have lower melting points when supplied, since such supplied products are often mixtures of fatty alcohols having alkyl chain length distribution in which the main alkyl chain is cetyl, stearyl or behenyl group.

Generally, in the mixture, the weight ratio of cetyl alcohol to stearyl alcohol is from about 1:9 to 9:1, alternatively from about 1:4 to about 4:1, alternatively from about 1:2.3 to about 1.5:1.

When using higher level of total cationic surfactant and high melting point fatty compounds, the mixture has the weight ratio of cetyl alcohol to stearyl alcohol of from about 1:1 to about 4:1, alternatively from about 1:1 to about 2:1, alternatively from about 1.2:1 to about 2:1, in view of maintaining acceptable consumer usage. It may also provide more conditioning on damaged part of the hair.

### Plasticizer

The first and/or the second layer may optionally comprise from about 1 wt % to about 25 wt % plasticizer, in one embodiment from about 3 wt % to about 20 wt % plasticizer, in one embodiment from about 5 wt % to about 15 wt % plasticizer. by weight of the layer.

When present in the Structures, non-limiting examples of suitable plasticizing agents include polyols, copolyols, polycarboxylic acids, polyesters and dimethicone copolyols.

Examples of useful polyols include, but are not limited to, glycerin, diglycerin, propylene glycol, ethylene glycol, butylene glycol, pentylene glycol, cyclohexane dimethanol, hexane diol, polyethylene glycol (200-600), sugar alcohols such as sorbitol, manitol, lactitol, isosorbide, glucamine, N-methylglucamine and other mono- and polyhydric low molecular weight alcohols (e.g., C₂-C₈ alcohols); mono di- and oligo-saccharides such as fructose, glucose, sucrose, maltose, lactose, and high fructose corn syrup solids and ascorbic acid.

Examples of polycarboxylic acids include, but are not limited to citric acid, maleic acid, succinic acid, polyacrylic acid, and polymaleic acid.

Examples of suitable polyesters include, but are not limited to, glycerol triacetate, acetylated-monoglyceride, diethyl phthalate, triethyl citrate, tributyl citrate, acetyl triethyl citrate, acetyl tributyl citrate.

Examples of suitable dimethicone copolyols include, but are not limited to, PEG-12 dimethicone, PEG/PPG-18/18 dimethicone, and PPG-12 dimethicone.

Other suitable plasticizers include, but are not limited to, alkyl and allyl phthalates; napthalates; lactates (e.g., sodium, ammonium and potassium salts); sorbeth-30; urea; lactic acid; sodium pyrrolidone carboxylic acid (PCA); sodium hyraluronate or hyaluronic acid; soluble collagen; modified protein; monosodium L-glutamate; alpha & beta hydroxyl acids such as glycolic acid, lactic acid, citric acid, maleic acid and salicylic acid; glyceryl polymethacrylate; polymeric plasticizers such as polyquaterniums; proteins and amino acids such as glutamic acid, aspartic acid, and lysine; hydrogen starch hydrolysates; other low molecular weight esters (e.g., esters of C₂-C₁₀ alcohols and acids); and any other water soluble plasticizer known to one skilled in the art of the foods and plastics industries; and mixtures thereof.

EP 0283165 B1 discloses suitable plasticizers, including glycerol derivatives such as propoxylated glycerol.

### Optional Ingredients

The first and/or the second layer may comprise other optional ingredients that are known for use or otherwise useful in compositions, provided that such optional materials are compatible with the selected essential materials described herein, or do not otherwise unduly impair product performance.

Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1992.

Further non-limiting examples of such optional ingredients include preservatives, perfumes or fragrances, coloring agents or dyes, conditioning agents, hair bleaching agents, thickeners, moisturizers, emollients, pharmaceutical actives, vitamins or nutrients, sunscreens, deodorants, sensates, plant extracts, nutrients, astringents, cosmetic particles, absorbent particles, adhesive particles, hair fixatives, fibers, reactive agents, skin lightening agents, skin tanning agents, anti-dandruff agents, perfumes, exfoliating agents, acids, bases, humectants, enzymes, suspending agents, pH modifiers, hair colorants, hair perming agents, pigment particles, anti-acne agents, anti-microbial agents, sunscreens, tanning agents, exfoliation particles, hair growth or restorer agents, insect repellents, shaving lotion agents, co-solvents or other additional solvents, and similar other materials. Further non-limiting examples of optional ingredients include encapsulated perfumes, such as by □-cyclodetrins, polymer microcapsules, starch encapsulated accords and combinations thereof.

Suitable conditioning agents include high melting point fatty compounds, silicone conditioning agents and cationic conditioning polymers. Suitable materials are discussed in US 2008/0019935, US 2008/0242584 and US 2006/0217288.

### Physical Properties of the Dissolvable Solid Structure

For fibrous Structures, the Structure comprises a significant number of dissolvable fibers with an average diameter less than about 150 micron, alternatively less than about 100 micron, alternatively less than about 10 micron, and alternatively less than about 1 micron with a relative standard deviation of less than 100%, alternatively less than 80%, alternatively less than 60%, alternatively less than 50%, such as in the range of 10% to 50%, for example. As set forth herein, the significant number means at least 10% of all the dissolvable fibers, alternatively at least 25% of all the dissolvable fibers, alternatively at least 50% of all the dissolvable fibers, alternatively at least 75% of all the dissolvable fibers. The significant number may be at least 99% of all the dissolvable fibers. Alternatively, from about 50% to about 100% of all the dissolvable fibers may have an average diameter less than about 10 micron. The dissolvable fibers produced by the method of the present disclosure have a significant number of dissolvable fibers with an average diameter less than about 1 micron, or sub-micron fibers. In an embodiment, Dissolvable Solid Structure may have from about 25% to about 100% of all the dissolvable fibers with an average diameter less than about 1 micron, alternatively from about 35% to about 100% of all the dissolvable fibers with an average diameter less than about 1 micron, alternatively from about 50% to about 100% of all the dissolvable fibers with an average diameter less than about 1 micron, and alternatively from about 75% to about 100% of all the dissolvable fibers with an average diameter less than about 1 micron.

The percent porosity of the dissolvable solid Structure is at least about 25%, alternatively at embodiment at least about 50%, alternatively at least about 60%, alternatively at least about 70% and alternatively at least about 80%. The porosity of the dissolvable solid Structure is not more than about 99%, alternatively not more than about 98%, alternatively not more than about 95%, and alternatively not more than about 90%. Porosity of a Structure is determined according to the procedure set forth in the definition of “porosity” above.

A range of effective sizes of pores can be accommodated. The pore size distribution through the Structure cross-section may be symmetric or asymmetric.

The Structure can be flexible and have a distance to maximum force value of from about 6 mm to about 30 mm. The distance to maximum force value from about 7 mm to about 25 mm, alternatively from about 8 mm to about 20 mm, and alternatively from about 9 mm to about 15 mm.

The Structure can be characterized in one aspect by its Specific Surface Area. The Structure can have a Specific Surface Area of from about 0.03 m²/g to about 0.25 m²/g, alternatively from about 0.035 m²/g to about 0.22 m²/g, alternatively from about 0.04 m²/g to about 0.19 m²/g, and alternatively from about 0.045 m²/g to about 0.16 m²/g.

The Structure can be a flat, flexible Structure in the form of a pad, a strip, or tape and having a thickness of from about 0.5 mm to about 10 mm, alternatively from about 1 mm to about 9 mm, alternatively from about 2 mm to about 8 mm, and alternatively from about 3 mm to about 7 mm as measured by the below methodology. The Structure can be a sheet having a thickness from about 5 mm to about 6.5 mm. Alternatively two or more sheets are combined to form a Structure with a thickness of about 5 mm to about 10 mm.

The Structure can have a basis weight of from about 200 grams/m² to about 2,000 grams/m², alternatively from about 400 g/m² to about 1,200 g/m², alternatively from about 600 g/m² to about 2,000 g/m², and alternatively from about 700 g/m² to about 1,500 g/m².

The Structure can have a dry density of from about 0.08 g/cm³ to about 0.40 g/cm³, alternatively from about 0.08 g/cm³ to about 0.38 g/cm³, alternatively from about 0.10 g/cm³ to about 0.25 g/cm³, and alternatively from about 0.12 g/cm³ to about 0.20 g/cm³.

### METHOD OF MANUFACTURING—Fibrous Structures

### Non-Limiting Example of Method for Making Fibrous Elements, Especially for the First Layer

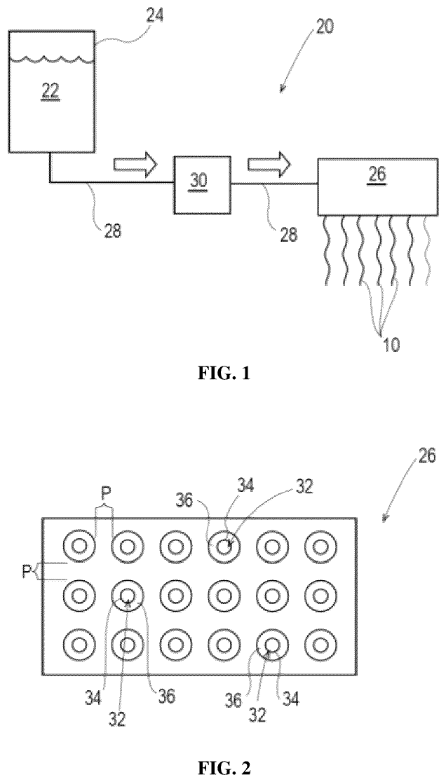

The fibrous elements, for example filaments, may be made as shown in FIGS. 1 and 2. As shown in FIGS. 1 and 2, a method 20 for making a fibrous element 10, for example filament, comprises the steps of:
a. providing a fibrous element-forming composition 22, such as from a tank 24, and
b. spinning the fibrous element-forming composition 22, such as via a spinning die 26, into one or more fibrous elements 10, such as filaments.

The fibrous element-forming composition may be transported via suitable piping 28, with or without a pump 30, between the tank 24 and the spinning die 26. In one example, a pressurized tank 24, suitable for batch operation is filled with a suitable fibrous element-forming composition 22 for spinning. A pump 30, such as a Zenith®, type PEP II, having a capacity of 5.0 cubic centimeters per revolution (cc/rev), manufactured by Colfax Corporation, Zenith Pumps Division, of Monroe, N.C., USA may be used to facilitate transport of the fibrous element-forming composition 22 to a spinning die 26. The flow of the fibrous element-forming composition 22 from the pressurized tank 24 to the spinning die 26 may be controlled by adjusting the number of revolutions per minute (rpm) of the pump 30. Pipes 28 are used to connect the pressurized tank 24, the pump 30, and the spinning die 26 in order to transport (as represented by the arrows) the fibrous element-forming composition 22 from the tank 24 to the pump 30 and into the die 26.

As shown in FIGS. 1 and 2, the spinning die 26 may comprise a plurality of fibrous element-forming holes 32 that include a melt capillary 34 encircled by a concentric attenuation fluid hole 36 through which a fluid, such as air, passes to facilitate attenuation of the fibrous element-forming composition 22 into a fibrous element 10 as it exits the fibrous element-forming hole 32.

In one example, the spinning die 26 shown in FIG. 2 has two or more rows of circular extrusion nozzles (fibrous element-forming holes 32) spaced from one another at a pitch P of about 1.524 millimeters (about 0.060 inches). The nozzles have individual inner diameters of about 0.305 millimeters (about 0.012 inches) and individual outside diameters of about 0.813 millimeters (about 0.032 inches). Each individual nozzle comprises a melt capillary 34 encircled by an annular and divergently flared orifice (concentric attenuation fluid hole 36) to supply attenuation air to each individual melt capillary 34. The fibrous element-forming composition 22 extruded through the nozzles is surrounded and attenuated by generally cylindrical, humidified air streams supplied through the orifices to produce fibrous elements 10.

Attenuation air can be provided by heating compressed air from a source by an electrical-resistance heater, for example, a heater manufactured by Chromalox, Division of Emerson Electric, of Pittsburgh, Pa., USA. An appropriate quantity of steam was added to saturate or nearly saturate the heated air at the conditions in the electrically heated, thermostatically controlled delivery pipe. Condensate was removed in an electrically heated, thermostatically controlled, separator.

The embryonic fibrous elements are dried by a drying air stream having a temperature from about 149° C. (about 300° F.) to about 315° C. (about 600° F.) by an electrical resistance heater (not shown) supplied through drying nozzles and discharged at an angle of about 90° relative to the general orientation of the embryonic fibrous elements being spun. The dried fibrous elements may be collected on a collection device, such as a belt or fabric, in one example a belt or fabric capable of imparting a pattern, for example a non-random repeating pattern to a fibrous structure formed as a result of collecting the fibrous elements on the belt or fabric. The addition of a vacuum source directly under the formation zone may be used to aid collection of the fibrous elements on the collection device. The spinning and collection of the fibrous elements produce a fibrous structure comprising inter-entangled fibrous elements, for example filaments.

In one example, during the spinning step, any volatile solvent, such as water, present in the fibrous element-forming composition 22 is removed, such as by drying, as the fibrous element 10 is formed. In one example, greater than 30% and/or greater than 40% and/or greater than 50% of the weight of the fibrous element-forming composition's volatile solvent, such as water, is removed during the spinning step, such as by drying the fibrous element 10 being produced.

### Non-Limiting Example of Method for Making Filament-Forming Composition, Especially for the Second Layer

The filament-forming composition may be made by any suitable process so long as the filament-forming composition is suitable for making the article.

In one example, one or more active agents, for example hair conditioning active agents such as cationic surfactants and high melting point fatty compounds, are added (in the absence of free water) to a metal beaker and heated to a temperature sufficient to melt the active agents, for example 80° C. The active agents are melted and optionally agitated until they form a homogeneous fluid.

After melting the active agents, other ingredients, for example one or more filament-forming materials, such as one or more structurants, are added to the homogeneous fluid of active agents. The other ingredients, when added, are stirred into the homogeneous fluid of active agents until the other ingredients are homogeneously dispersed throughout the homogeneous fluid of active agents and/or are homogeneously dissolved within the homogeneous fluid of active agents. This all occurs while maintaining the homogeneous fluid of active agents at a temperature of at least the melting point of the lowest melting point active agent, for example 80° C.

The filament-forming composition may then be used to make fibrous elements and/or fibrous structures and/or articles.

### Non-Limiting Example of Method for Making Fibrous Elements, Especially for the Second Layer

The fibrous elements may be made by any suitable process. A non-limiting example of a suitable process for making the fibrous elements is described below.

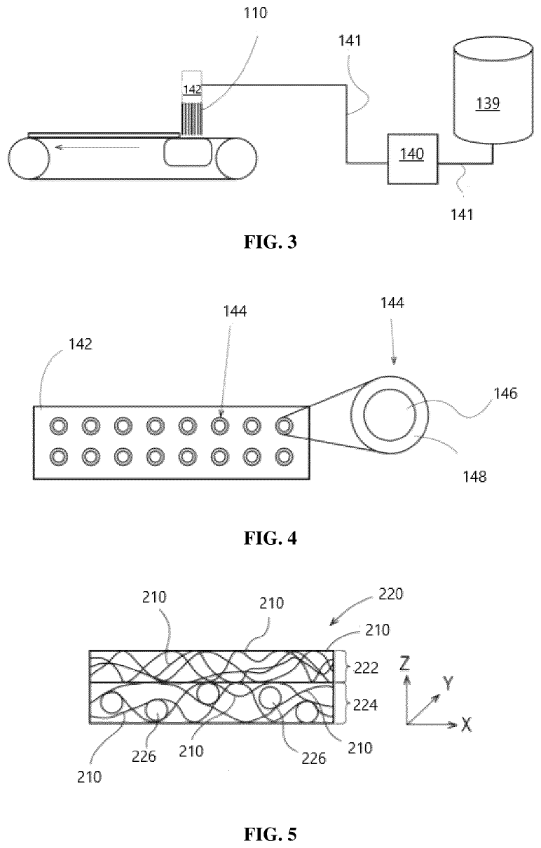

As shown in FIGS. 3 and 4, the fibrous elements may be made as follows. Fibrous elements may be formed by means of a small-scale apparatus, a schematic representation of which is shown in FIGS. 3 and 4. A pressurized tank 139, suitable for batch operation is filled with a suitable filament-forming composition. A pump 140 such as a Zenith®, type PEP II, having a capacity of 5.0 cubic centimeters per revolution (cm³/rev), manufactured by Parker Hannifin Corporation, Zenith Pumps division, of Sanford, N.C., USA may be used to facilitate transport of the filament-forming composition via pipes 141 to a spinning die 142. The flow of the filament-forming composition from the pressurized tank 139 to the spinning die 142 may be controlled by adjusting the number of revolutions per minute (rpm) of the pump 140. Pipes 141 are used to connect the pressurized tank 139, the pump 140, and the spinning die 142.

The spinning die 142 shown in FIG. 3 has several rows of circular extrusion nozzles (fibrous element-forming holes 44) spaced from one another at a pitch P of about 1.524 millimeters (about 0.060 inches). The nozzles have individual inner diameters of about 0.305 millimeters (about 0.012 inches) and individual outside diameters of about 0.813 millimeters (about 0.032 inches). Each individual nozzle is encircled by an annular and divergently flared orifice (concentric attenuation fluid hole 148 to supply attenuation air to each individual melt capillary 146. The filament-forming composition extruded through the nozzles is surrounded and attenuated by generally cylindrical, humidified air streams supplied through the orifices.

In one example, as shown in FIGS. 3 and 4, a method for making a fibrous element 110 comprises the steps of:
a. providing a filament-forming composition comprising one or more filament-forming materials, and optionally one or more active agents; and
b. spinning the filament-forming composition, such as via a spinning die 142, into one or more fibrous elements, such as filaments 110, comprising the one or more filament-forming materials and optionally, the one or more active agents. The one or more active agents may be releasable from the fibrous element when exposed to conditions of intended use.

As shown in FIG. 4, the spinning die 142 may comprise a plurality of fibrous element-forming holes 144 that include a melt capillary 146 encircled by a concentric attenuation fluid hole 148 through which a fluid, such as air, passes to facilitate attenuation of the filament-forming composition into a fibrous element, for example a filament 110 as it exits the fibrous element-forming hole 144.

Attenuation air can be provided by heating compressed air from a source by an electrical-resistance heater, for example, a heater manufactured by Chromalox, Division of Emerson Electric, of Pittsburgh, Pa., USA. An appropriate quantity of steam was added to saturate or nearly saturate the heated air at the conditions in the electrically heated, thermostatically controlled delivery pipe. Condensate is removed in an electrically heated, thermostatically controlled, separator.

The embryonic fibrous elements are quenched by a cooling air stream having a temperature from about 5° C. (about 41° F.) to about 40° C. (about 104° F.) by an chilling air handler (not shown) supplied through cooling air ducts and discharged at an angle of about 90° relative to the general orientation of the embryonic fibrous elements being extruded. The dried embryonic fibrous elements are collected on a collection device, such as, for example, a movable foraminous belt or patterned collection belt. The addition of a vacuum source directly under the formation zone may be used to aid collection of the fibers.

### Non-Limiting Example of Method for Making Article

As shown in FIG. 5, another example of an article 220, for example a fibrous structure of the present disclosure comprises a first fibrous structure layer 222 comprising a plurality of fibrous elements, for example filaments 210, and a second fibrous structure layer 224 comprising a plurality of fibrous elements, for example filaments 210, for example active agent-containing filaments, and a plurality of particles 226, for example active agent-containing particles, dispersed, in this case randomly, in the x, y, and z axes, throughout the second fibrous structure layer 224. Alternatively, in another example, the plurality of particles 226, for example active agent-containing particles, may be dispersed in an irregular pattern or a non-random, repeating pattern within the second fibrous structure layer 224. Like above, a similar article comprising two plies of fibrous structure comprising a first fibrous structure ply 222 comprising a plurality of fibrous elements, for example filaments 210, and a second fibrous structure ply 224 comprising a plurality of fibrous elements, for example filaments 210, for example active agent-containing filaments, and a plurality of particles 226, for example active agent-containing particles, dispersed, in this case randomly, in the x, y, and z axes, throughout the second fibrous structure ply 224. Alternatively, in another example, the plurality of particles 226, for example active agent-containing particles, may be dispersed in an irregular pattern or a non-random, repeating pattern within the second fibrous structure ply 224.

In one example, in a multi-ply article, one or more fibrous structure plies may be formed and/or deposited directly upon an existing ply of fibrous structure to form a multi-ply fibrous structure. The two or more existing fibrous structure plies may be combined, for example via thermal bonding, gluing, embossing, aperturing, rodding, rotary knife aperturing, die cutting, die punching, needle punching, knurling, pneumatic forming, hydraulic forming, laser cutting, tufting, and/or other mechanical combining process, with one or more other existing fibrous structure plies to form the multi-ply article.

### Method of Use

The dissolvable solid substrates described herein may be used for cleaning and/or treating hair, hair follicles, skin, teeth, and the oral cavity. The method for treating these consumer substrates may comprise the steps of: a) applying an effective amount of the Structure to the hand, b) wetting the Structure with water to dissolve the solid, c) applying the dissolved material to the target consumer substrate such as to clean or treat it, and d) rinsing the diluted treatment composition from the consumer substrate. These steps can be repeated as many times as desired to achieve the desired cleansing and or treatment benefit.

A method useful for providing a benefit to hair, hair follicles, skin, teeth, and/or the oral cavity, includes the step of applying a composition according to the first embodiment to these target consumer substrates in need of regulating.

Alternatively, a useful method for regulating the condition of hair, hair follicles, skin, teeth, the oral cavity, includes the step of applying one or more compositions described herein to these target consumer substrates in need of regulation.

The amount of the composition applied, the frequency of application and the period of use will vary widely depending upon the purpose of application, the level of components of a given composition and the level of regulation desired. For example, when the composition is applied for whole body or hair treatment, effective amounts generally range from about 0.5 grams to about 10 grams, alternatively from about 1.0 grams to about 5 grams, and alternatively from about 1.5 grams to about 3 grams.

### Product Types and Articles of Commerce

Non-limiting examples of products that utilize the dissolvable solid structures include hand cleansing substrates, teeth cleaning or treating substrates, oral cavity substrates, hair shampoo or other hair treatment substrates, body cleansing substrates, shaving preparation substrates, personal care substrates containing pharmaceutical or other skin care active, moisturizing substrates, sunscreen substrates, chronic skin benefit agent substrates (e.g. vitamin-containing substrates, alpha-hydroxy acid-containing substrates, etc.), deodorizing substrates, fragrance-containing substrates, and so forth.

Alternatively, the dissolvable solid structure is a personal care product, alternatively hair care product, alternatively rinse-off hair care product, alternatively rinse-off hair care product containing non-sulfate surfactant.

Described herein is an article of commerce comprising one or more dissolvable solid structures described herein, and a communication directing a consumer to dissolve the Structure and apply the dissolved mixture to hair, hair follicles, skin, teeth, the oral cavity, to achieve a benefit to the target consumer substrate, a rapidly lathering foam, a rapidly rinsing foam, a clean rinsing foam, and combinations thereof. The communication may be printed material attached directly or indirectly to packaging that contains the dissolvable solid structure or on the dissolvable solid structure itself. Alternatively, the communication may be an electronic or a broadcast message that is associated with the article of manufacture. Alternatively, the communication may describe at least one possible use, capability, distinguishing feature and/or property of the article of manufacture.

### Test Methods

### Basis Weight Measurement

In general, basis weight of a material or article (including the dissolvable solid structure) is measured by first cutting the sample to a known area, using a die cutter or equivalent, then measuring & recording the weight of the sample on a top-loading balance with a minimum resolution of 0.01 g, then finally by calculating the basis weight as follows:

$$Basis Weight ( g / {m}^{2} ) = weight of basis weight pad ( g )$$

$$Basis Weight ( \frac{g}{{m}^{2}} ) = \frac{Weight of pad ( g ) \times 10 , 000 \frac{c {m}^{2}}{{m}^{2}}}{area of pad ( {cm}^{2} )}$$

Suitable pad sample sizes for basis weight determination are >10 cm² and should be cut with a precision die cutter having the desired geometry. If the dissolvable solid structure to be measured is smaller than 10 cm², a smaller sampling area can be sued for basis weight determination with the appropriate changes to calculation.

In the present examples, basis weight was calculated based on the full dissolvable solid structure having a known area of 17.28 cm². Thus, the basis weight calculation becomes:

$$( \frac{g}{{m}^{2}} ) = \frac{Weight of pad ( g ) \times 10 , 000 \frac{c {m}^{2}}{{m}^{2}}}{17.28 {cm}^{2}}$$

### Hand Dissolution Test Method

### Materials Needed:

Dissolvable solid structures to be tested: 3-5 dissolvable solid structure s (finished product samples) are tested so that an average of the number of strokes for each if the individual dissolvable solid structure samples is calculated and recorded as the Average Hand Dissolution value for the dissolvable solid structure. For this method, the entire consumer saleable or consumer use dissolvable solid structure is tested. If the entire consumer saleable or consumer use dissolvable solid structure has a footprint greater than 50 cm², then first cut the dissolvable solid structure to have a footprint of 50 cm².
Nitrile Gloves
   10 cc syringe
   Plastic Weigh boat (˜3 in ×3 in)
   100 mL Glass beaker
   Water (City of Cincinnati Water or equivalent having the following properties: Total Hardness=155 mg/L as CaCO2; Calcium content=33.2 mg/L; Magnesium content=17.5 mg/L; Phosphate content=0.0462 mg/L)
Water used is 7 gpg hardness and 40° C.+/−5° C.

### Protocol:

1. Add 80 mL of water to glass beaker. Add 300-500 ml of water to glass beaker.
2. Heat water in beaker until water is at a temperature of 40° C.+/−5° C.
3. Transfer 10 mL of the water from the beaker into the weigh boat via the syringe.
4. Within 10 seconds of transferring the water to the weigh boat, place dissolvable solid structure sample in palm of gloved hand (hand in cupped position in non-dominant hand to hold dissolvable solid structure sample).
5. Using dominant hand, add water quickly from the weigh boat to the dissolvable solid structure sample and allow to immediately wet for a period of 5-10 seconds.
6. Rub with opposite dominant hand (also gloved) in 2 rapid circular strokes.
7. Visually examine the dissolvable solid structure sample in hand after the 2 strokes. If dissolvable solid structure sample is completely dissolved, record number of strokes=2 Dissolution Strokes. If not completely dissolved, rub remaining dissolvable solid structure sample for 2 more circular strokes (4 total) and observe degree of dissolution. If the dissolvable solid structure sample contains no solid pieces after the 2 additional strokes, record number of strokes=4 Dissolution Strokes. If after the 4 strokes total, the dissolvable solid structure sample still contains solid pieces of un-dissolved dissolvable solid structure sample, continue rubbing remaining dissolvable solid structure sample in additional 2 circular strokes and check if there are any remaining solid pieces of dissolvable solid structure sample after each additional 2 strokes until dissolvable solid structure sample is completely dissolved or until reaching a total of 30 strokes, whichever comes first. Record the total number of strokes. Record 30 Dissolution Strokes even if solid dissolvable solid structure sample pieces remain after the maximum of 30 strokes.
8. Repeat this process for each of the additional 4 dissolvable solid structure samples.
9. Calculate the arithmetic mean of the recorded values of Dissolution Strokes for the 5 individual dissolvable solid structure samples and record as the Average Hand Dissolution Value for the dissolvable solid structure. The Average Hand Dissolution Value is reported to the nearest single Dissolution Stroke unit.

### Fibrous Structures-Fiber Diameter

For fibrous Structures, the diameter of dissolvable fibers in a sample of a web is determined by using a Scanning Electron Microscope (SEM) or an Optical Microscope and image analysis software. A magnification of 200 to 10,000 times is chosen such that the fibers are suitably enlarged for measurement. When using the SEM, the samples are sputtered with gold or a palladium compound to avoid electric charging and vibrations of the fibers in the electron beam. A manual procedure for determining the fiber diameters is used from the image (on monitor screen) taken with the SEM or the optical microscope. Using a mouse and a cursor tool, the edge of a randomly selected fiber is sought and then measured across its width (i.e., perpendicular to fiber direction at that point) to the other edge of the fiber. A scaled and calibrated image analysis tool provides the scaling to get actual reading in microns (μm). Several fibers are thus randomly selected across the sample of the web using the SEM or the optical microscope. At least two specimens from the web (or web inside a product) are cut and tested in this manner. Altogether at least 100 such measurements are made and then all data are recorded for statistical analysis. The recorded data are used to calculate average (mean) of the fiber diameters, standard deviation of the fiber diameters, and median of the fiber diameters. Another useful statistic is the calculation of the amount of the population of fibers that is below a certain upper limit. To determine this statistic, the software is programmed to count how many results of the fiber diameters are below an upper limit and that count (divided by total number of data and multiplied by 100%) is reported in percent as percent below the upper limit, such as percent below 1 micron diameter or %-submicron, for example. We denote the measured diameter (in microns) of an individual circular fiber as di.

In case the fibers have non-circular cross-sections, the measurement of the fiber diameter is determined as and set equal to the hydraulic diameter which is four times the cross-sectional area of the fiber divided by the perimeter of the cross of the fiber (outer perimeter in case of hollow fibers). The number-average diameter, alternatively average diameter is calculated as, dₙᵤₘ

$$\frac{{\sum }_{i = 1}^{n} {d}_{i}}{n}$$

### Combinations

1. A dissolvable solid structure comprising:
   a. A first layer comprising from about 20% to about 100%, alternatively from about 50% to about 100%, alternatively from about 90% to about 100%, alternatively from about 95% to about 100%, alternatively from about 99% to about 100%, by weight of the first layer by weight of the first layer of a first water-soluble polymer,
   b. A second layer comprising: from about 1% to about 50%, alternatively from about 1% to about 20%, alternatively from about 1% to about 10%, alternatively from about 2% to about 6%, alternatively from about 3% to about 5%, by weight of the second layer, of a second water-soluble polymer; a high melting point fatty material having a carbon chain length C12-C22 or mixtures thereof, wherein the melting point is above 25° C.; and a cationic surfactant.
2. The dissolvable solid structure of the preceding feature, wherein the first layer has a Scaled Fail Tensile Energy Absorption (Scaled Fail TEA) of from about 10 (g/cm) to about 185 (g/cm), alternatively form about 20 (g/cm) to about 180 (g/cm), alternatively from about 25 (g/cm) to about 175 (g/cm).
3. The dissolvable solid structure of the preceding feature, wherein the first water-soluble polymer is selected from the group consisting of:
   (i) a polyvinyl alcohol having a molecular weight of from about 23,000 g/mol to about 45,000 g/mol, alternatively from about 25,000 g/mol to about 40,000 g/mol, alternatively from about 26,000 g/mol to about 35,000 g/mol;
   (ii) a mixture of polyvinyl alcohols, wherein the mixture has an average molecular weight as a mixture of from about 23,000 g/mol to about 45,000 g/mol, alternatively from about 25,000 g/mol to about 40,000 g/mol, alternatively from about 26,000 g/mol to about 35,000 g/mol;
   (iii) a thermal bonded starch with or without a plasticizer, wherein molecular weight of from about 1,000,000 g/mol to about 50,000,000 g/mol, alternatively from about 2,000,0000 g/mol to about 40,000,000 g/mol;
   (iv) Polyvinylpyrrolidone, its copolymer, or mixtures thereof;
   (v) Poly vinyl oxazoline, its copolymer, or mixtures thereof;
   (vi) Poly 2-ethyl-2oxazoline, its copolymer, or mixtures thereof; and
   (vii) Polyvinylcaprolactam, its copolymer, or mixtures thereof.
4. The dissolvable solid structure of any of the preceding features, wherein the first water-soluble polymer is selected from (ii) the mixture of polyvinyl alcohols.
5. The dissolvable solid structure of any of the preceding features, wherein the second water-soluble polymer is selected from the group consisting of: polyvinylpyrrolidone, alternatively a polyvinylpyrrolidone copolymer; polydimethylacrylamide, alternatively a polydimethylacrylamide copolymer; a polyvinyl oxazoline, alternatively poly 2-ethyl-2-oxazoline; a polyvinylcaprolactam copolymer, alternatively polyvinyl caprolactam and combinations thereof.
6. The dissolvable solid structure of any of the preceding features, wherein the first layer comprises: the first water-soluble polymer; and an ingredient incompatible to at least one of the above ingredients in the second layer;
wherein second layer comprises: the second water-soluble polymer; a high melting point fatty material having a carbon chain length C12-C22 or mixtures thereof, wherein the melting point is above 25° C.; and a cationic surfactant.
7. The dissolvable solid structure of any of the preceding features, wherein the first layer has an aesthetic feature selected from the group consisting of printing, embossing, texture, colored and mixtures thereof.
8. The dissolvable solid structure of any of the preceding features, wherein the first layer contains substantially free of a surfactant.
9. The dissolvable solid structure of any of the preceding features, wherein at least one of the first and second layers has a particulate, alternatively the second layer has the particulate.
10. The dissolvable solid structure of any of the preceding features, wherein the Dissolvable Solid Structure dissolves in less than 30 strokes of the Hand Dissolution Method, alternatively wherein the Dissolvable Solid Structure dissolves in less than 20 strokes of the Hand Dissolution Method, alternatively wherein the Dissolvable Solid Structure dissolves in less than 15 strokes of the Hand Dissolution Method.
11. The dissolvable solid structure of any of the preceding features, wherein the dissolvable solid structure is a personal care product, alternatively hair care product, alternatively rinse-off hair care product, alternatively rinse-off hair care product containing non-sulfate surfactant.

### Non-Limiting Examples

The compositions illustrated in the following Examples illustrate specific embodiments of the composition but are not intended to be limiting thereof. Other modifications can be undertaken by the skilled artisan without departing from the spirit and scope of this invention. These exemplified embodiments of the composition as described herein provide enhanced conditioning benefits to the hair.

All exemplified amounts are listed as weight percent and exclude minor materials such as diluents, preservatives, color solutions, imagery ingredients, botanicals, and so forth, unless otherwise specified. All percentages are based on weight unless otherwise specified.

### First Layer—Examples

| Raw Material | F-Ex 1 | F-Ex 2 | F-Ex 3 | F-Ex 4 | F-Ex 5 |
|---|---|---|---|---|---|
| Polyvinyl alcohol 403¹ | 100 | 50 | 87.8 | | 0 |
| Polyvinyl alcohol 420h² | 0 | 50 | 0 | | 0 |
| Polyvinyl alcohol 505³ | 0 | 0 | 11.7 | | 0 |
| Polyvinyl alcohol 205⁴ | 0 | 0 | 0 | 90 | 0 |
| Sorbitol | 0 | 0 | 0 | 10 | 0 |
| Purity gum Starch with | 0 | 0 | 0 | 0 | 100 |
| thermal bonding | | | | | |
| Oleic Acid | 0 | 0 | 0.5 | 0 | 0 |
| Molecular weight or | 24.5 kDa | 97.8 kDa | 27.5 kDa | 48.1 kDa | 13000 kDa |
| Average molecular weight | | | | | |
| of the polymer (Mw kDa) | | | | | |
| Scaled Fail TEA (g/cm) | 39 | 51 | 71 | 170 | 151 |

¹Selvol 403 from Kurraray
²Selvol 420h from Kurraray
³Selvol 505 from Kurraray
⁴Selvol 205 from Kurraray

### First Layer Comparative Example 1

| Raw Material | Comparative example 1 |
|---|---|
| Purity Gum | 90 |
| Sorbitol | 10 |
| Mw (kDa) | 13000 kDa |
| Scaled Fail | 3.5 |
| TEA (g/cm) | |

### First Layer Comparative Example 2

| Raw Material | Comparative example 2 |
|---|---|
| PVOH 10-98 | 100 |
| Mw (kDa) | 50 kDa |
| Scaled Fail | 194.6 |
| TEA (g/cm) | |

### Second Layer—Examples

| Raw Material | S-Ex 1 | S-Ex 2 | S-Ex 3 | S-Ex 4 | S-Ex 5 | S-Ex 6 | S-Ex 7 | S-Ex 8 | S-Ex 9 | S-Ex 10 |
|---|---|---|---|---|---|---|---|---|---|---|
| Distilled Water | 2.3 | 2.5 | 2.3 | 2.4 | 2.3 | 2.5 | 2.5 | 2.3 | 2.5 | 2.5 |
| Behentrimonium | 20.6 | 4.1 | 32.8 | 4.1 | 0.0 | 0.0 | 32.8 | 32.8 | 32.8 | 20.4 |
| Methosulfate | | | | | | | | | | |
| Behentrimonium | 0.0 | 0.0 | 0.0 | 0.0 | 20.6 | 20.4 | 0.0 | 0.0 | 0.0 | 0.0 |
| Chloride | | | | | | | | | | |
| Stearyl Alcohol | 45.2 | 55.0 | 38.0 | 51.3 | 45.2 | 24.6 | 34.3 | 30.7 | 17.5 | 24.6 |
| 1-Hexadecanol | 18.5 | 23.1 | 16.4 | 21.6 | 18.5 | 39.0 | 14.9 | 13.6 | 26.7 | 39.0 |
| Lauroyl Methyl | 9.2 | 12.2 | 2.1 | 12.3 | 9.2 | 9.2 | 12.3 | 12.3 | 12.2 | 9.2 |
| Glucamide | | | | | | | | | | |
| Polyvinyl | 4.1 | 3.1 | 8.3 | 8.3 | 4.1 | 4.2 | 3.1 | 8.3 | 8.3 | 4.2 |
| pyrrolidone | | | | | | | | | | |
| (PVP K120 from | | | | | | | | | | |
| Ashland) | | | | | | | | | | |

### Dissolvable Solid Structure Examples

| | D-Ex 1 | D-Ex 2 | D-Ex 3 | D-Ex 4 | D-Ex 5 | D-Ex 6 |
|---|---|---|---|---|---|---|
| First layer | F-Ex 1 | F-Ex 2 | F-Ex 3 | F-Ex3 | F-Ex 4 | F-Ex 5 |
| composition | | | | | | |
| First layer | 50 | 100 | 50 | 75 | 100 | 100 |
| (gsm) | | | | | | |
| Second layer | S-Ex 3 | S-Ex 4 | S-Ex 1 | S-Ex 6 | S-Ex 10 | S-Ex 7 |
| composition | | | | | | |
| Second layer | 350 | 350 | 350 | 350 | 500 | 350 |
| (gsm) | | | | | | |
| Ratio of the | 0.14 | 0.29 | 0.14 | 0.21 | 0.2 | 0.29 |
| First Layer to | | | | | | |
| the second | | | | | | |
| Layer | | | | | | |

The dissolvable solid structures of D-Ex.1 to D-Ex. 6 are examples using First layer examples F-Ex. 1 to F-Ex. 5 and some of the Second layer examples such as S-Ex.1. These examples provide improved flexibility in a composition, especially allowing its lower contents of polymers at least in one of the layers, while keeping a certain structural strength during manufacturing and easiness to cut during and after manufacturing.

When using First Layer Comparative Example 1, the dissolvable solid structure could not maintain a desired sheet form during the manufacturing. When using First Layer Comparative Example 2, the dissolvable solid structure was hard to cut during and after manufacturing.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dissolvable solid structure comprising:
a. a first layer comprising filaments; wherein the filaments comprise from about 20% to 100%, by weight of the first layer, of a first water-soluble polymer comprising a starch having a molecular weight of from about 1,000,000 g/mol to about 50,000,000 g/mol;
wherein the first layer is free of a surfactant;
b. a second layer comprising filaments; wherein the filaments comprise:
from about 1% to about 50%, by weight of the second layer, of a second water-soluble polymer;
a high melting point fatty material comprising an average carbon chain length from C12-C22 and the melting point is above 25° C.; and
a cationic surfactant.

2. The dissolvable solid structure of claim 1, wherein the starch is a thermal bonded starch.

3. The dissolvable solid structure of claim 2, wherein the first layer further comprises from about 1% to about 25% of a plasticizer.

4. The dissolvable solid structure of claim 3, wherein the plasticizer is chosen from polyols, copolyols, polycarboxylic acids, polyesters, dimethicone copolyols, or mixtures thereof.

5. The dissolvable solid structure of claim 4, wherein the plasticizer comprises a polyester chosen from glycerol triacetate, acetylated-monoglyceride, diethyl phthalate, triethyl citrate, tributyl citrate, acetyl triethyl citrate, acetyl tributyl citrate, or mixtures thereof.

6. The dissolvable solid structure of claim 4, wherein the plasticizer comprises a polycarboxylic acid chosen from citric acid, maleic acid, succinic acid, polyacrylic acid, polymaleic acid, or mixtures thereof.

7. The dissolvable solid structure of claim 4, wherein the plasticizer comprises a dimethicone copolyol chosen from PEG-12 dimethicone, PEG/PPG-18/18 dimethicone, and PPG-12 dimethicone, or mixtures thereof.

8. The dissolvable solid structure of claim 3, wherein the plasticizer is chosen from glycerin, diglycerin, propylene glycol, ethylene glycol, butylene glycol, pentylene glycol, cyclohexane dimethanol, hexane diol, polyethylene glycol, sugar alcohols, isosorbide, glucamine, N-methylglucamine, mono-low molecular weight alcohols, polyhydric low molecular weight alcohols, monosaccharides, disaccharides, oligosaccharides, ascorbic acid, or mixtures thereof.

9. The dissolvable solid structure of claim 1, wherein the starch has a molecular weight of from about 2,000,000 g/mol to about 40,000,000 g/mol.

10. The dissolvable solid structure of claim 1, wherein the first layer has a Scaled Fail Tensile Energy Absorption of from about 10 g/cm to about 185 g/cm.

11. The dissolvable solid structure of claim 10, wherein the first layer has a Scaled Fail Tensile Energy Absorption of about 20 g/cm to about 180 g/cm.

12. The dissolvable solid structure of claim 11, wherein the first layer has a Scaled Fail Tensile Energy Absorption alternatively from about 25 g/cm to about 175 g/cm.

13. The dissolvable solid structure of claim 1, wherein the first layer comprises from about 50% to about 100%, by weight of the first layer, of the first water-soluble polymer.

14. The dissolvable solid structure of claim 1, wherein the second layer comprises from about 1% to about 20%, by weight of the second layer, of the second water-soluble polymer.

15. The dissolvable solid structure of claim 1, wherein the second water-soluble polymer is chosen from polyvinylpyrrolidone, a polyvinylpyrrolidone copolymer, polydimethylacrylamide, a polydimethylacrylamide copolymer, a polyvinyl oxazoline polymer, poly 2-ethyl-2-oxazoline, a polyvinylcaprolactam copolymer, polyvinyl caprolactam, or mixtures thereof.

16. The dissolvable solid structure of claim 15, wherein the second water-soluble polymer is selected from the group consisting of polyvinylpyrrolidone, a polyvinylpyrrolidone copolymer, or mixtures thereof.

17. The dissolvable solid structure of claim 1, wherein the first layer further comprises an ingredient incompatible with at least one of the ingredients in the second layer.

18. The dissolvable solid structure of claim 1, wherein at least one of the first or second layers comprises a particulate.

19. The dissolvable solid structure of any of claim 18, wherein the second layer comprises the particulate.

20. The dissolvable solid structure of claim 1, wherein the dissolvable solid structure dissolves in less than 15 strokes of the Hand Dissolution Method.
